(19) United States
(12) Patent Application Publication
(10) Pub. No.: US 2021/0128772 A1
(43) Pub. Date: May 6, 2021

(54) NEBULIZER DIFFUSER

(71) Applicant: LIFE & LIVING INTERNATIONAL LIMITED, Kowloon Bay (HK)

(72) Inventor: Jaw-Chyi Shieh, Kowloon Bay (HK)

(21) Appl. No.: 16/672,588

(22) Filed: Nov. 4, 2019

(51) Int. Cl. A61L 9/12; A61M 11/00; A61L 9/14

(52) U.S. Cl. CPC A61L 9/122; A61M 11/005; A61L 2209/12; A61M 2205/33; A61L 9/14

(57) ABSTRACT

A nebulizer diffuser comprises a base, a nebulizer module, a power supply unit, at least one fluid container module, and a cover. The base comprises a conducting chamber comprising a first through hole, wherein the conducting chamber is for holding a first fluid; an accommodation space, wherein the conducting chamber is located above the accommodation space. The nebulizer module comprises an oscillator and a controller which manages the operation of the oscillator, wherein the oscillator is attached to the first through hole and the controller is disposed in the accommodation space. The power supply unit is disposed in the accommodation space and electrically connected to the nebulizer module. The at least one fluid container module comprises a fluid tank separably located on the conducting chamber and the fluid tank is for holding a second fluid. The cover separably covers on the fluid tank and an air chamber is formed thereinside.

### TECHNICAL FIELD

The present inventive concept relates to a nebulizer diffuser, and more particularly, to a nebulizer diffuser with at least one modular fluid container.

### BACKGROUND

Essential oils are commonly used in the practice of aromatherapy which is a form of alternative medicine. Inhaling the aromas from essential oils may be good for one's health. Some are useful to soothe and heal, others relieve stress and tension and still others may promote restful sleep.

Essential oil diffusers are developed to vaporize aromatic ingredients in the essential oils and release the aromatic ingredients into the atmosphere. Some diffusers even combine with nebulizers. The technology inside a nebulizer is to have an electronic vibrating element in contact with a liquid reservoir and generate some vapor mist. Water or other carrier liquid with drops of essential oils is in the liquid reservoir and the aromatic ingredients can be released with the vapor mist.

Essential oil diffuser needs to be cleaned once the essential oils are changed, since the scents from different oils may interfere with each other. Besides, it is important to clean an essential oil diffuser due to oil residue stuck inside the diffuser, which affects how well the diffuser works and the residue may be harmful to one's health.

### SUMMARY

Exemplary embodiments of the general inventive concept provide a nebulizer diffuser. The nebulizer diffuser comprises a base, a nebulizer module, a power supply unit and at least one fluid container module.

The base comprises a conducting chamber and an accommodation space. The conducting chamber comprises a first through hole, wherein the conducting chamber is for holding a first fluid and wherein the conducting chamber is located above the accommodation space.

The nebulizer module comprises an oscillator and a controller which manages the operation of the oscillator, wherein the oscillator is attached to the first through hole and the controller is disposed in the accommodation space.

The power supply unit is disposed in the accommodation space and electrically connected to the nebulizer module.

The at least one fluid container module comprises a fluid tank and a cover. The fluid tank is separably located on the conducting chamber, wherein the fluid tank has a conical bottom having a predetermined central surface substantially in the center of the conical bottom and the fluid tank is for holding a second fluid; and the cover separably covers on the fluid tank and forming an air chamber thereinside.

In an exemplary embodiment of the present inventive concept, the base further comprises a first side wall and at least one first restricted part, and the at least one first restricted part is disposed on the outside of the first side wall; the at least one fluid container module further comprises a second side wall and at least one second restricted part, and the at least one second restricted part is disposed on the outside of the second side wall; and wherein the at least one first restricted part and the at least one second restricted part are separably engaged with each other.

In an exemplary embodiment of the present inventive concept, the top further comprises at least one third restricted part. The at least one third restricted part is disposed on the top of the cover, wherein the at least one third restricted part and the at least one second restricted part are correspondingly engageable with each other.

In an exemplary embodiment of the present inventive concept, the predetermined central surface of the conical bottom is substantially located above the oscillator.

In another exemplary embodiment in the present inventive concept, the predetermined central surface of the conical bottom is substantially on the first fluid in the conducting chamber.

In another exemplary embodiment in the present inventive concept, the nebulizer diffuser further comprises a fan. The fan is disposed in the accommodation space, wherein the power supply unit is further electrically connected to the fan.

In an exemplary embodiment of the present inventive concept, the conducting chamber further comprises a third side wall, at least one first recess and at least one first opening corresponding to the at least one first recess.

The at least one first recess is formed on the third side wall and the at least one first opening is disposed on the third side wall and provides access from the at least one first recess to the accommodation space through the at least one first opening.

In an exemplary embodiment of the present inventive concept, the fluid tank further comprises at least one second recess and at least one second opening corresponding to the at least one second recess.

The at least one second recess is formed on the second side wall and corresponding to the at least one first recess; and the at least one second opening is disposed on the second side wall and provides access for the air chamber to the at least one first recess through the at least one second opening.

In an exemplary embodiment of the present inventive concept, the cover further comprises a collecting hood disposed on the inner surface of the top of the cover. The collecting hood comprises a mouth and at least one second through hole thereon, wherein the collecting hood is configured to allow the mouth towards the fluid tank.

In an exemplary embodiment of the present inventive concept, the cover further comprises at least one third through hole thereon.

In an exemplary embodiment of the present inventive concept, the cover further comprises at least one guiding part disposed inside the cover at the at least one third through hole thereon.

In another exemplary embodiment in the present inventive concept, the nebulizer diffuser further comprises an input unit.

The input unit is disposed on the base, wherein the input unit is for receiving an instruction and controls the operations of the controller and the power supply unit according to the instruction.

In an exemplary embodiment of the present inventive concept, the nebulizer diffuser further comprises a timer, wherein the input unit is further for setting the timer; and wherein the controller is for managing the operation of the oscillator according to the set timer.

In another exemplary embodiment in the present inventive concept, the nebulizer diffuser further comprises a luminous element and the base further comprises a non-opaque part. The luminous element is disposed in the accommodation space; and wherein the controller is for managing the operation of the luminous element.

In another exemplary embodiment in the present inventive concept, the nebulizer diffuser further comprises a luminous element and the base further comprises a non-opaque part. The luminous element is disposed in the accommodation space; and wherein the controller is further for managing the operation of the luminous element according to the instruction received by the input unit.

In another exemplary embodiment in the present inventive concept, the nebulizer diffuser further comprises a luminous element and the base further comprises a non-opaque part. The luminous element is disposed in the accommodation space; and wherein the controller is further for controlling the timer according to the instruction received by the input unit which makes the controller manage the operation of the luminous element according to the set timer.

The nebulizer diffuser of the present inventive concept comprises a conducting chamber and at least one fluid container module separate from the nebulizer chamber, which is easier for users to clean the nebulizer diffuser of the present inventive concept. Besides, the at least one fluid container module and the cover of the nebulizer diffuser of the present inventive concept respectively comprises a second restricted part and a third restricted part which are correspondingly engageable with each other, which makes the nebulizer diffuser of the present inventive concept to be expandable to includes multiple fluid container modules.

### BRIEF DESCRIPTION OF DRAWINGS

FIG. 1 is a schematic exploded view showing a structure according to an exemplary embodiment of the present inventive concept;

FIG. 2 is a schematic exploded view showing a part of the structure of another exemplary embodiment of the present inventive concept;

FIG. 3 is a schematic diagram showing the modules of according to an exemplary embodiment of the present inventive concept;

FIG. 4(A) is a schematic view showing a structure of an exemplary embodiment of the present inventive concept; FIG. 4(B) is a schematic cross-sectional view, as section A-A in FIG. 4(A), showing the structure of according to an exemplary embodiment of the present inventive concept.

### DETAILED DESCRIPTION

Reference will now be made in detail to the embodiments of the present general inventive concept, examples of which are illustrated in the accompanying drawings, wherein like reference numerals refer to the like elements throughout. The embodiments are described below in order to explain the present general inventive concept while referring to the figures. Also, while describing the present general inventive concept, detailed descriptions about related well-known functions or configurations that may diminish the clarity of the points of the present general inventive concept are omitted.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed description of the invention. Thus, the terms used herein have to be defined based on the meaning of the terms together with the description throughout the specification.

Also, when a part “includes” or “comprises” an element, unless there is a particular description contrary thereto, the part can further include other elements, not excluding the other elements. In the following description, terms such as “unit” and “module” indicate a unit to process at least one function or operation, wherein the unit and the block may be embodied as hardware or software or embodied by combining hardware and software.

Hereinafter, one or more exemplary embodiments of the present general inventive concept will be described in detail with reference to accompanying drawings.

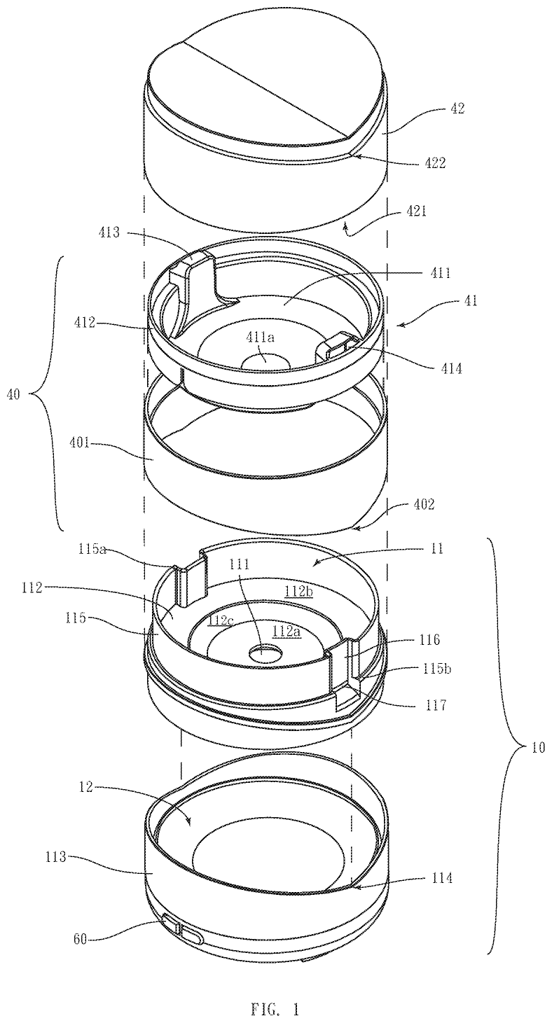

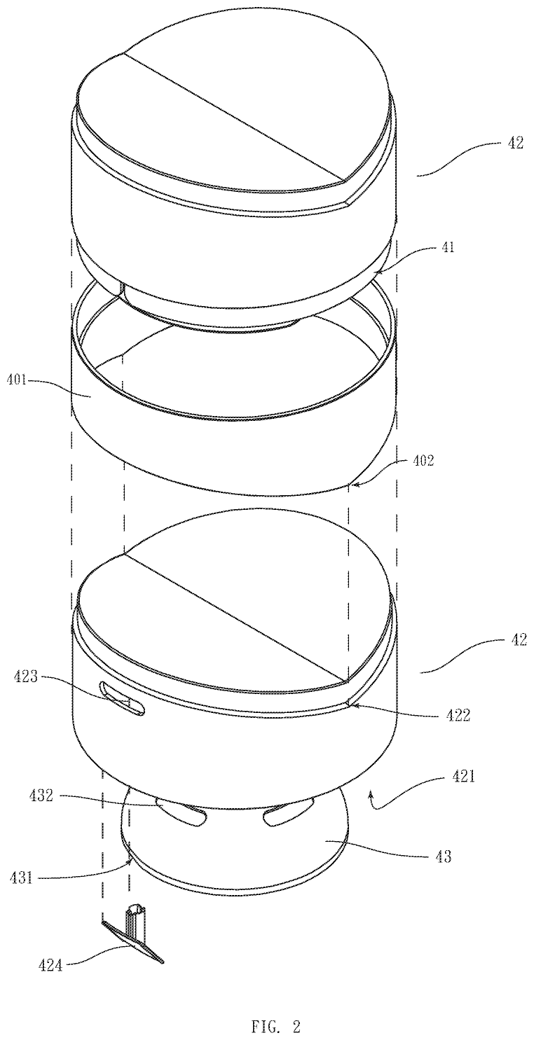

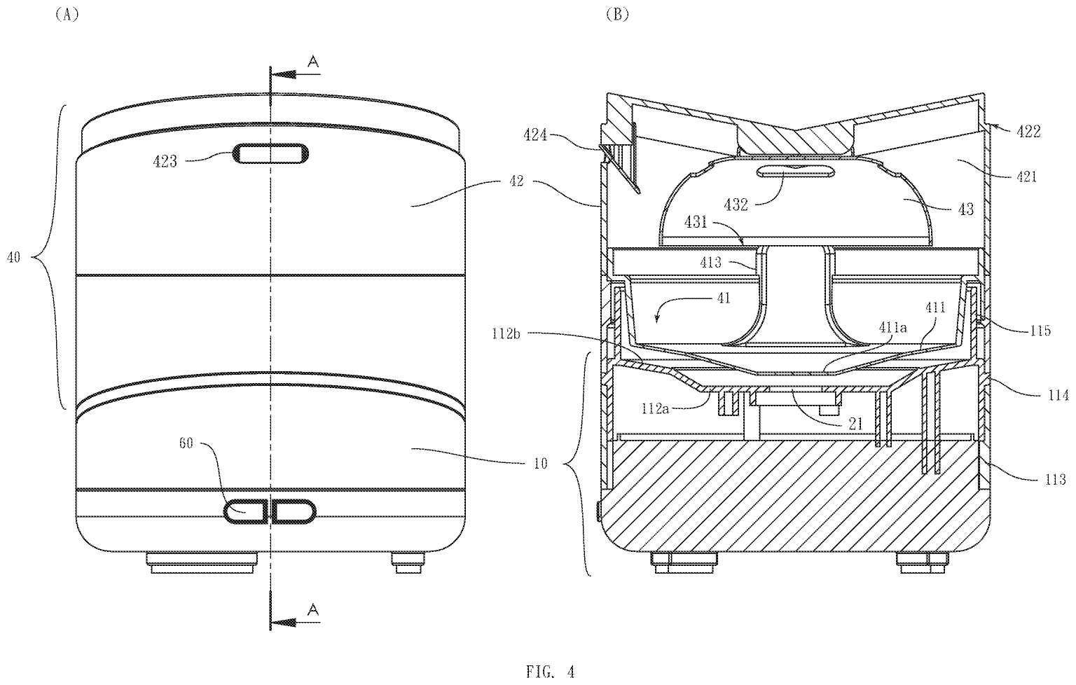

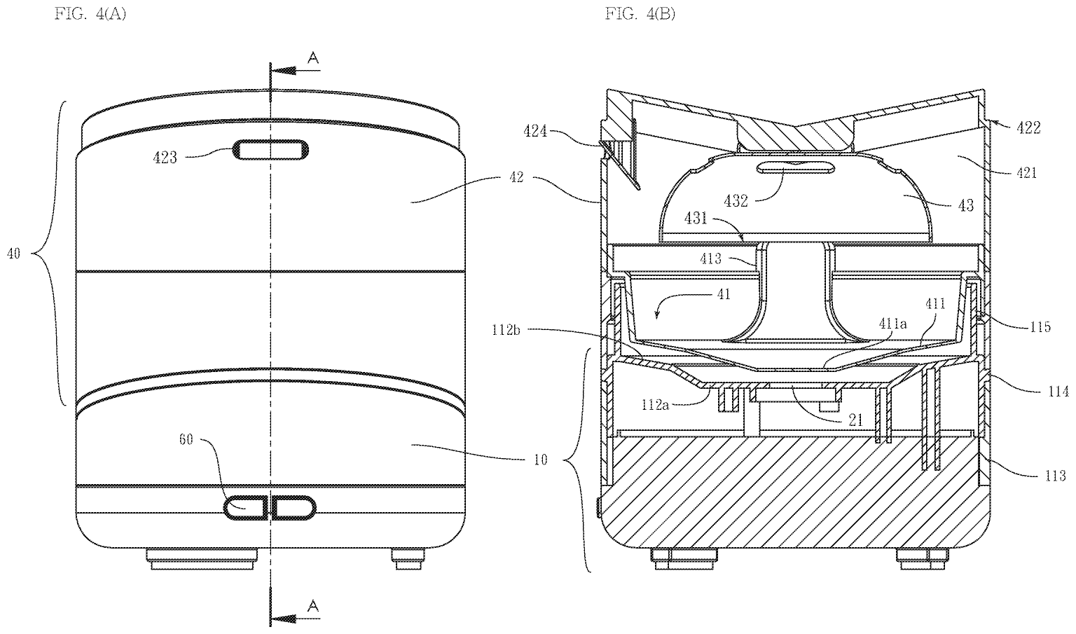

The general inventive concept of the present invention provides a nebulizer diffuser. Referring to FIGS. 1 to 4, the nebulizer diffuser may comprise a base 10, a nebulizer module 20, a power supply unit 30 and at least one fluid container module 40.

According to an exemplary embodiment of the present inventive concept, the base 10 may comprise a conducting chamber 11 and an accommodation space 12. The conducting chamber 11 may comprise a first through hole 111. In an exemplary embodiment, the conducting chamber 11 may be used to hold a first fluid and wherein the conducting chamber 11 is located above the accommodation space 12.

In an exemplary embodiment, the first fluid may be water, such as distilled water or tap water.

In an exemplary embodiment, the conducting chamber 11 may have a bottom 112, and the first through hole 111 may be disposed on the bottom 112. Preferably, the bottom 112 of the conducting chamber 11 may further have a first stage 112a and a second stage 112b around the first stage 112a with a substantially parallel surface in the first stage 112a. More preferably, the second stage 112b is higher than the first stage 112a and the first through hole 111 may be disposed on the substantially parallel surface.

In another exemplary embodiment, the second stage 112b may be in the shape of frustum cone and connected to the first stage 112a by another truncated conical surface 112c. In this embodiment, the edge between the second stage 112b and the truncated conical surface 112c may be used as a water level mark for the first fluid.

Referring to FIGS. 1 to 4, according to an exemplary embodiment of the present inventive concept, the nebulizer module 20 may comprise an oscillator 21 and a controller 22. The controller 22 may manage the operation of the oscillator 21, wherein the oscillator 21 may be attached to the first through hole 111 and the controller 22 may be disposed in the accommodation space 12.

In an exemplary embodiment, the nebulizer module 20 may be an ultrasonic wave nebulizer module. In this exemplary embodiment, the oscillator 21 may generate a high frequency ultrasonic wave to cause the vibration of a piezoelectric element (not shown in the figure) in the oscillator 21. The vibrating element may be in contact with a liquid reservoir and its high frequency vibration is sufficient to produce a vapor mist.

In another exemplary embodiment, the nebulizer module 20 may be a VMT nebulizer module. In this exemplary embodiment, a mesh (not shown in the figure) may be on the oscillator 21. The oscillator 21 may vibrate the mesh to make the liquid into very small particles which come out through the mesh and form fine mist.

According to the present inventive concept, the nebulizer module 20 may include any suitable nebulizer, for example but not limited to, an ultrasonic wave nebulizer or a VMT (vibrating mesh technology) nebulizer.

According to an exemplary embodiment of the present inventive concept, the power supply unit 30 may be disposed in the accommodation space 12 and electrically connected to the nebulizer module 20.

The power supply unit 30 may be a power adaptor receiving electric power from such as an electrical outlet, energy storage devices such as batteries or fuel cells, generators or alternators, solar power converters, or another power supply. The power supply unit 30 may be used to providing electric power to electrical loads in nebulizer diffuser of the present inventive concept.

According to an exemplary embodiment of the present inventive concept, the at least one fluid container module 40 may comprise a fluid tank 41 and a cover 42. The fluid tank 41 may be separably located on the conducting chamber 11 and used to hold a second fluid; and the cover 42 may separably cover on the fluid tank 41 and forming an air chamber 421 thereinside.

In an exemplary embodiment, the second fluid may be water, such as distilled water or tap water, with some drops of essential oils.

According to an exemplary embodiment of the present inventive concept, the fluid tank 41 may have a bottom 411. Preferably, the bottom 411 of the fluid tank 41 may have a lower center 411a. More preferably, the bottom 411 of the fluid tank 41 may be in the shape of an inverted truncated cone.

According to an exemplary embodiment of the present inventive concept, the lower center 411a of the bottom 411 of the fluid tank 41 may be substantially located above the oscillator 21. In some exemplary embodiments, the lower center 411a of the bottom 411 of the fluid tank 41 may not be substantially in contact with the oscillator 21.

According to another exemplary embodiment of the present inventive concept, the lower center 411a of the bottom 411 of the fluid tank 41 is substantially on the first fluid in the conducting chamber 11. In some exemplary embodiments, the lower center 411a of the bottom 411 of the fluid tank 41 may be substantially in contact with the first fluid in the conducting chamber 11. In this exemplary embodiment, the vibrating oscillator 21 vibrates the first fluid which causes the vibration of the bottom 411 of the fluid tank 41 in contact with the first fluid. The vibrating fluid tank 41 may vibrate the second fluid therein and generate vapor mist from the second fluid.

According to the present inventive concept, the lower center 411a of the bottom 411 of the fluid tank 41 may be made of a first material and the rest of the bottom 411 of the fluid tank 41 may be made of a second material.

The first material may be any material which is waterproof and lighter, such as acrylonitrile butadiene styrene, polylactic acid, polyvinyl alcohol, polypropylene or the like. Alternatively, the first material may be any material which is waterproof and heat resistant, such as metal, ceramic or the like.

The second material may be the same as the first material. Alternatively, the second material may be any material which is waterproof and lighter, such as acrylonitrile butadiene styrene, polylactic acid, polyvinyl alcohol, polypropylene or the like.

According to an exemplary embodiment of the present inventive concept, the base 10 may further comprise a first side wall 113 and at least one first restricted part 114. The at least one first restricted part 114 may be disposed on the outside of the first side wall 113. In this exemplary embodiment, the at least one fluid container module 40 may further comprise a second side wall 401 and at least one second restricted part 402. The at least one second restricted part 402 may be disposed on the outside of the second side wall 401. Wherein the at least one first restricted part 114 and the at least one second restricted part 402 may be separably engaged with each other.

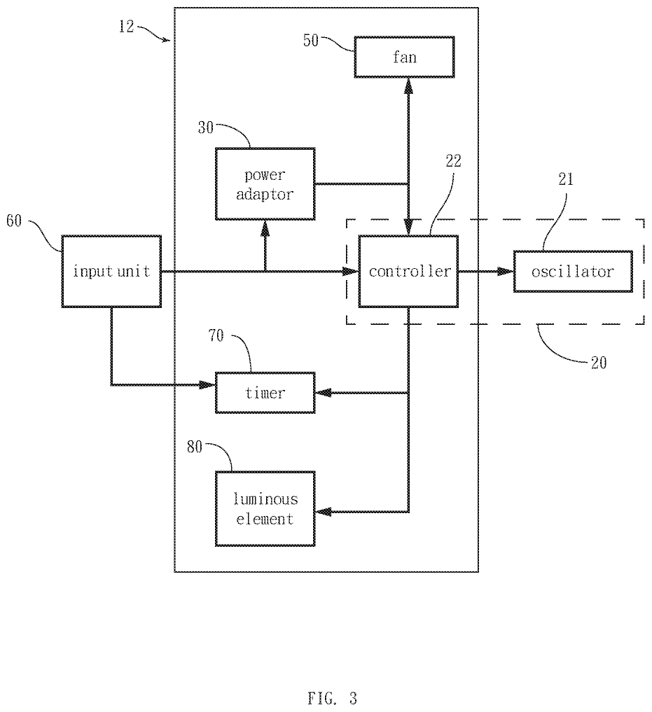

Referring to FIG. 2, according to an exemplary embodiment of the present inventive concept, the cover 42 may further comprise at least one third restricted part 422. The at least one third restricted part 422 may be disposed on the top of the cover 42, wherein the at least one third restricted part 422 and the at least one second restricted part 402 are correspondingly engageable with each other.

Referring to FIGS. 1 to 4, according to an exemplary embodiment of the present inventive concept, the nebulizer diffuser may further comprise a fan 50. The fan 50 may be disposed in the accommodation space 12, wherein the power supply unit 30 may be further electrically connected to the fan 50.

According to an exemplary embodiment of the present inventive concept, the conducting chamber 11 may further comprise a third side wall 115, at least one first recess 116 and at least one first opening 117 corresponding to the at least one first recess 116.

In an exemplary embodiment of the present inventive concept, the at least one first recess 116 may be formed on the third side wall 115 and the at least one first opening 117 may be disposed on the third side wall 115 and the at least one first opening 117 may provide access from the at least one first recess 116 to the accommodation space 12 through the at least one first opening 117.

According to an exemplary embodiment of the present inventive concept, the fluid tank 41 may further comprise at least one second recess 413 and at least one second opening 414 corresponding to the at least one second recess 413, wherein the at least one second recess 413 may be formed on the second side wall 401 and corresponding to the at least one first recess 116; and the at least one second opening 414 may be disposed on the second side wall 401 and provides access for the air chamber 421 to the at least one first recess 116 through the at least one second opening 414.

According to another exemplary embodiment of the present inventive concept, the second side wall 401 may further comprise an inner wall 412, wherein the inner wall 412 may be inside around the second side wall 401.

In this exemplary embodiment, the at least one second recess 413 may be formed on the inner wall 412 and corresponding to the at least one first recess 116; and the at least one second opening 414 may be disposed on the inner wall 412 and the at least one second opening 414 may provide access for the air chamber 421 to the at least one first recess 116 through the at least one second opening 414.

According to an exemplary embodiment of the present inventive concept, when the fluid tank 41 locates on the conducting chamber 11, the at least one first recess 116 and the at least one second recess 413 may be corresponding to each other.

In a preferable exemplary embodiment of the present inventive concept, the at least one first recess 116 may be formed from the top 115a of the third side wall 115 towards the bottom 115b of the third side wall 115; and the at least first opening 117 may be disposed near the bottom of the at least one first recess 116. The least one second recess 413 may be formed from the top of the inner wall 412 towards the bottom of the inner wall 412; and the at least one second opening 414 may be disposed at the top of the at least one second recess 413. The least one second recess 413 may be formed from the top of the second side wall 401 towards the bottom of the second side wall 401; and the at least one second opening 414 may be disposed at the top of the at least one second recess 413. In this exemplary embodiment, when the fluid tank 41 locates on the conducting chamber 11, the at least one first recess 116, the at least one second recess 413 and the second side wall 401 may together form a channel which provide access from the accommodation space 12 to the air chamber 421 through the at least one first opening 117 via the channel to the at least second opening 414.

In a preferable exemplary embodiment of the present inventive concept, the fan 50 may be disposed in the accommodation space 12 to generate airflows. The airflows may flow through the at least first opening 117 via the channel formed by the at least one first recess 116, the at least one second recess 413 and the second side wall 401, through the at least second opening 414 to the air chamber 421. By doing so, the airflow may be guided and concentrated by the channel.

Referring to FIGS. 1 to 2, according to an exemplary embodiment of the present inventive concept, the cover 42 may further comprise a collecting hood 43. The collecting hood 43 may be disposed on the inner surface of the top of the cover 42. The collecting hood 43 may comprise a mouth 431 and at least one second through hole 432 thereon, wherein the collecting hood 43 may be configured to allow the mouth 431 towards the fluid tank 41.

According to the present inventive concept, the collecting hood 43 may have a hemisphere surface or be in the shape of a frustum.

In an exemplary embodiment of the present inventive concept, the mouth 431 of the collecting hood 43 may collect the vapor mist from the second fluid in the fluid tank 41, which makes the vapor mist concentrated and deflected to flow through the second through hole 432 on the collecting hood.

According to an exemplary embodiment of the present inventive concept, the cover 42 may further comprise at least one third through hole 423 thereon. According to the present inventive concept, the air in the air chamber 421 may flow out through the third through hole 423.

According to a preferable exemplary embodiment of the present inventive concept, the cover 42 may further comprise at least one guiding part 424 disposed inside the cover 42 at the at least one third through hole 423 thereon. The air in the air chamber 421 may be guided by the guiding part 424 and flow out through the third through hole 423.

Referring to FIGS. 1 to 3, according to an exemplary embodiment of the present inventive concept, the nebulizer diffuser may further comprise an input unit 60. The input unit 60 may be disposed on the base 10, wherein the input unit 60 may be used to receive an instruction and manage the operations of the controller 22 and the power supply unit 30 according to the instruction.

According to an exemplary embodiment of the present inventive concept, the nebulizer diffuser may further comprise a timer 70. In this exemplary embodiment, the input unit 60 may further be used to set the timer 70; and the controller 22 may be used to manage the operation of the oscillator 21 according to the set timer 70.

Specifically, the nebulizer diffuser of the exemplary embodiment of the present incentive concept may be set in sleep mode. The input unit 60 may be used to receive a time-instruction and the timer 70 may be set for a desired time period by the time-instruction received by the input unit 60 and the controller 22 may be used to manage the operation of the oscillator 21 according to the set timer 70. For example, the controller 22 may turn on the oscillator 21 for the desired time period and turn the oscillator 21 off after the period of time.

According to an exemplary embodiment of the present inventive concept, the nebulizer diffuser may further comprise a luminous element 80 and the base 10 may further comprise a non-opaque part (not denoted in the figures). The luminous element 80 may be disposed in the accommodation space 12. Specifically, the luminous element 80 may emit light, and the light mat pass through the non-opaque part.

According to the present inventive concept, the luminous element 80 may be various lighting such as LED lighting, compact fluorescent lighting, incandescent lighting and the like.

According to the present inventive concept, the non-opaque part of the base 10 may be transparent or translucent. The non-opaque part may be made of clear, frosted, or ground glass; or clear, frosted, or ground acrylic; or the like, as long as all or some light would pass through the non-opaque part.

In an exemplary embodiment of the present inventive concept, the controller 22 may further be used to manage the operation of the luminous element 80.

In another exemplary embodiment of the present inventive concept, the controller 22 may further be used to manage the operation of the luminous element 80 according to the instruction received by the input unit 60.

In another exemplary embodiment in the present inventive concept, the controller 22 may further be used to control the timer 70 according to the instruction received by the input unit 60 which makes the controller 22 manage the operation of the luminous element 80 according to the set timer 70.

Specifically, the controller 22 may be used to turn on/off the luminous element 80.

Preferably, the input unit 60 may be used to receive a lighting-instruction and the controller 22 may further be used to manage the operation of the luminous element 80 according to the lighting-instruction. For example, the controller 22 may turn on/off the luminous element 80 or the luminous element 80 may be adjusted by the controller 22.

Preferably, the input unit 60 may be used to receive a time-instruction and the timer 70 may be set for a desired time period by the time-instruction received by the input unit 60 and the controller 22 may be used to manage the operation of the luminous element 80 according to the set timer 70. For example, the controller 22 may turn on the luminous element 80 for the desired time period and turn the luminous element 80 off after the period of time.

In an exemplary embodiment, the luminous element 80 may be set in dimming mode. For example, the timer 70 may be set for a desired time period by the time-instruction received by the input unit 60 and the controller 22 may be used to set the luminous element 80 in the dimming mode, which means the lights emitted from the luminous element 80 fade to dark according to the set timer 70.

The nebulizer diffuser of the present inventive concept comprises a conducting chamber 11 and at least one fluid container module 40 separate from the nebulizer chamber 11, which is easier for users to clean the nebulizer diffuser of the present inventive concept. By doing so, it lengthens the lifetime of the nebulizer diffuser and keeps the nebulizer diffuser running at its optimal efficiency.

Besides, the at least one fluid container module 40 and the cover 42 of the nebulizer diffuser of the present inventive concept respectively comprises a second restricted part 402 and a third restricted part 422 which are correspondingly engageable with each other, which allows the nebulizer diffuser of the present inventive concept to be expandable and to comprise multiple fluid container modules 40 and the scents of the essential oils in different fluid container modules would not interfere with each other, which is shown as in FIG. 2.

The various exemplary embodiments as described herein are merely used to describe the principle, characteristic, and effect of the present invention, but not to limit the scope of the present invention. Anyone with ordinary skills in the arts can modify or change the various exemplary embodiments without departing from the spirit and scope of the disclosure. Accordingly, all modifications and variations completed by those with ordinary skill in the art should fall within the scope of present invention defined by the appended claims. The scope of the present general inventive concept should be specified as the appended claims. OR: Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

What is claimed is:

1. A nebulizer diffuser, comprising:
a base, comprising:
a conducting chamber comprising a first through hole, wherein the conducting chamber is for holding a first fluid;
an accommodation space, wherein the conducting chamber is located above the accommodation space; and
a nebulizer module comprising an oscillator and a controller which manages the operation of the oscillator, wherein the oscillator is attached to the first through hole and the controller is disposed in the accommodation space;
a power supply unit disposed in the accommodation space and electrically connected to the nebulizer module; and
at least one fluid container module, comprising
a fluid tank separably located on the conducting chamber and the fluid tank is for holding a second fluid; and
a cover separably covering on the fluid tank and an air chamber is formed thereinside.

2. The nebulizer diffuser according to claim 1, wherein
the base further comprises
a first side wall; and
at least one first restricted part disposed on the outside of the first side wall;
the at least one fluid container module further comprises
a second side wall; and
at least one second restricted part disposed on the outside of the second side wall; and
wherein the at least one first restricted part and the at least one second restricted part are separably engaged with each other.

3. The nebulizer diffuser according to claim 2, wherein the cover further comprises at least one third restricted part which is disposed on the top of the cover, wherein the at least one third restricted part and the at least one second restricted part are correspondingly engageable with each other.

4. The nebulizer diffuser according to claim 1, wherein the conducting chamber further has a bottom and the first through hole is disposed thereon.

5. The nebulizer diffuser according to claim 4, wherein the bottom of the conducting chamber has a first stage and a second stage around the first stage with a substantially parallel surface in the first stage.

6. The nebulizer diffuser according to claim 5, wherein the second stage is higher than the first stage; and the first through hole is disposed on the substantially parallel surface.

7. The nebulizer diffuser according to claim 1, wherein the fluid tank has a bottom substantially on the first fluid in the conducting chamber.

8. The nebulizer diffuser according to claim 1, wherein the fluid tank has a bottom having a lower center, which is substantially located above the oscillator.

9. The nebulizer diffuser according to claim 8, wherein the lower center of the bottom of the fluid tank is substantially on the first fluid in the conducting chamber.

10. The nebulizer diffuser according to claim 8, wherein the lower center of the bottom of the fluid tank is made of a first material and the rest of the bottom of the fluid tank is made of a second material.

11. The nebulizer diffuser according to claim 2, further comprising,
a fan disposed in the accommodation space, wherein the power supply unit is further electrically connected to the fan.

12. The nebulizer diffuser according to claim 10, wherein the conducting chamber further comprises a third side wall, at least one first recess and at least one first opening corresponding to the at least one first recess,
wherein the at least one first recess is formed on the third side wall and the at least one first opening is disposed on the third side wall and provides access from the at least one first recess to the accommodation space through the at least one first opening.

13. The nebulizer diffuser according to claim 11, wherein the fluid tank further comprises at least one second recess and at least one second opening corresponding to the at least one second recess,
wherein the at least one second recess is formed on the second side wall and corresponding to the at least one first recess; and
the at least one second opening is disposed on the second side wall and provides access for the air chamber to the at least one first recess through the at least one second opening.

14. The nebulizer diffuser according to claim 1, wherein the cover further comprises a collecting hood disposed on the inner surface of the top of the cover and comprising a mouth and at least one second through hole thereon, wherein the collecting hood is configured to allow the mouth towards the fluid tank.

15. The nebulizer diffuser according to claim 1, wherein the cover further comprises at least one third through hole thereon.

16. The nebulizer diffuser according to claim 14, wherein the cover further comprises at least one guiding part disposed inside the cover at the at least one third through hole thereon.

17. The nebulizer diffuser according to claim 1, further comprising,
an input unit disposed on the base, wherein the input unit is for receiving an instruction and controls the operations of the controller and the power supply unit according to the instruction.

18. The nebulizer diffuser according to claim 16, further comprising,
a timer, wherein the input unit is further for setting the timer; and wherein the controller is for managing the operation of the oscillator according to the set timer.

19. The nebulizer diffuser according to claim 18, further comprising,
a luminous element disposed in the accommodation space; and
the base further comprises a non-opaque part, and
wherein the controller is further for managing the operation of the luminous element according to the instruction received by the input unit.

20. The nebulizer diffuser according to claim 19, further comprising,
a luminous element disposed in the accommodation space; and
the base further comprises a non-opaque part, and
wherein the controller is further for controlling the timer according to the instruction received by the input unit, which makes the controller manage the operation of the luminous element according to the set timer.

21. The nebulizer diffuser according to claim 1, further comprising,
a luminous element disposed in the accommodation space; and
the base further comprises a non-opaque part, and
wherein the controller is for managing the operation of the luminous element.
